(19) United States
(12) Plant Patent Application Publication
(10) Pub. No.: US 2008/0184413 P1
(43) Pub. Date: Jul. 31, 2008

(54) Griselinia plant named 'Green Hedgemaster'

(76) Inventor: Tom Johnson, Auckland (NZ)

Correspondence Address: MARK P. BOURGEOIS, 2851 Rockinghorse Lane, Niles, MI, 49120, US

(21) Appl. No.: 11/880,054

(22) Filed: Jul. 19, 2007

(30) Foreign Application Priority Data
Jul. 31, 2006 (NZ) TRM086

(51) Int. Cl. A01H 5/00

(52) U.S. Cl. PLT/226

(57) ABSTRACT

A new cultivar of Griselinia plant named ‘Green Hedgemaster’ that is characterized by flat rounded leaves, a semi-upright habit and medium green leaves.

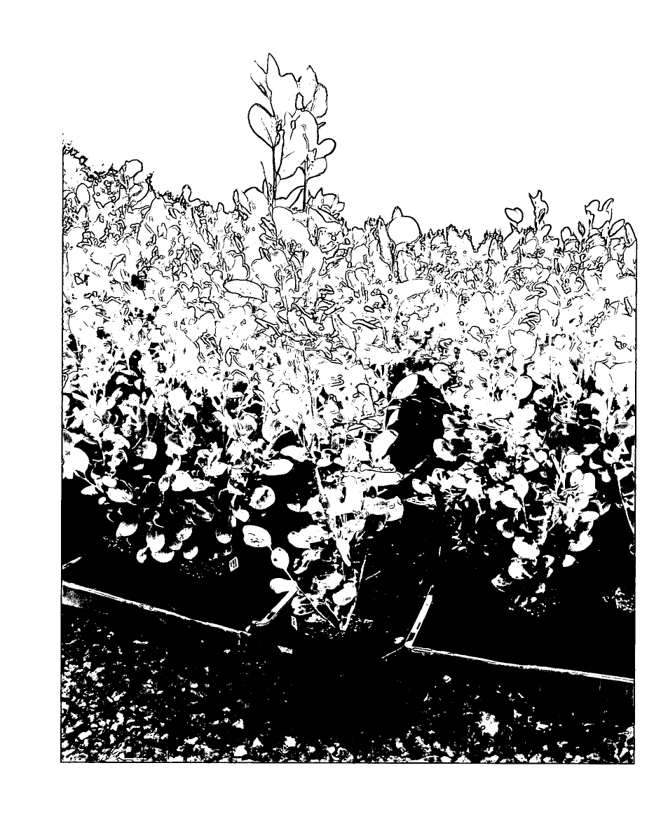

### BOTANICAL CLASSIFICATION

Griselinia littoralis

### VARIETY DENOMINATION

‘Green Hedgemaster’

### BACKGROUND OF THE INVENTION

The present invention relates to a new and distinct cultivar of Griselinia plant botanically known as Griselinia littoralis and hereinafter referred to by the cultivar name ‘Green Hedgemaster’.

The new cultivar is a naturally occurring branch mutation of Griselinia littoralis ‘Variegata’ (not patented). ‘Green Hedgemaster’ was discovered by the inventor in a cultivated area of Auckland, New Zealand in 2002.

Asexual reproduction by tip cuttings of the new cultivar ‘Green Hedgemaster’ was first performed in 2004 in Auckland, New Zealand. Since that time, under careful observation, the unique characteristics of the new cultivar have been uniform, stable and reproduced true to type in successive generations of asexual reproduction.

### SUMMARY OF THE INVENTION

The following represent the distinguishing characteristics of the new Griselinia cultivar ‘Green Hedgemaster’.
1. Griselinia ‘Green Hedgemaster’ exhibits flat rounded leaves.
2. Griselinia ‘Green Hedgemaster’ exhibits a semi-upright habit.
3. Griselinia ‘Green Hedgemaster’ exhibits medium green leaves.

The new cultivar ‘Green Hedgemaster’ is distinguishable from the parent plant by the following characteristics:

1. ‘Green Hedgemaster’ has solid green leaves. The leaves of ‘Variegata’ are variegated.

2. ‘Green Hedgemaster’ has longer leaves.

3. ‘Green Hedgemaster’ has an orbicular leaf shape. The leaves of ‘Vareigata’ are elliptic.

4. ‘Green Hedgemaster’ is more vigorous.

5. ‘Green Hedgemaster’ has a shorter crop time.

The closest comparison cultivars are Griselinia ‘Broadway Mint’ (not patented) and Griselinia ‘Whenauapai’ (not patented).

The new cultivar ‘Green Hedgemaster’ is distinguishable from ‘Broadway Mint’ by the following characteristics:

1. ‘Green Hedgemaster’ has shorter leaves.

2. ‘Green Hedgemaster’ has an orbicular leaf shape. The leaves of ‘Broadway Mint are Oval.

3. ‘Green Hedgemaster’ is more vigorous.

The new cultivar ‘Green Hedgemaster’ is distinguishable from ‘Whenauapai’ by the following characteristics:

1. ‘Green Hedgemaster’ has longer leaves.

2. ‘Green Hedgemaster’ has an orbicular leaf shape. The leaves of ‘Whenauapai are oval.

3. ‘Green Hedgemaster’ is more vigorous.

4. ‘Green Hedgemaster’ has darker green leaves.

### BRIEF DESCRIPTION OF THE DRAWING

The accompanying photograph illustrates the distinguishing traits of Griselinia ‘Green Hedgemaster’. The plant in the photograph shows an overall view of a 2 year old plant. The photograph was taken using conventional techniques and although colors may appear different from actual colors due to light reflectance it is as accurate as possible by conventional photographic techniques.

### BOTANICAL DESCRIPTION OF THE PLANT

The following is a detailed description of the new Griselinia cultivar named ‘Green Hedgemaster’. Data was collected in Auckland, New Zealand from 2 year old field grown plants. The time of year was Winter in the Southern Hemisphere and the average temperature range was 12 to 18 degrees Centigrade during the day and 4 to 11 degrees Centigrade at night. No photoperiodic treatments or growth retardants were used. Color determinations are in accordance with The Royal Horticultural Society Colour Chart 2001 edition, except where general color terms of ordinary dictionary significance are used. The growing requirements are similar to the species. ‘Green Hedgemaster’ has not been tested under all possible conditions and phenotypic differences may be observed with variations in environmental, climatic, and cultural conditions, however, without any variance in genotype.
Botanical classification: Griselinia littoralis ‘Green Hedgemaster’.
Use: Ornamental perennial shrub.
Parentage: ‘Green Hedgemaster’ is a naturally occurring branch mutation of Griselinia ‘Variegata’.
Vigor: Vigourous.
Growth rate: 1.0 meter per year.
Growth habit: Semi upright.
Plant shape: Rounded.
Suitable container size: 2.0 liter container.
Height: Average 8.0 m in height.
Width: Average 4.0 m in width.
Hardiness: Tolerant to −7 degrees Centigrade.
Propagation: Tip cuttings.
Time to initiate roots: Approximately 50 days in the Summer and 60 days in the Winter.
Time to produce a rooted cutting: Approximately 84 days in the Summer and 95 days in the Winter.
Root system: Fine and fibrous.
Lateral branches:
   Quantity.—Approximately 60.
   Length.—Average 0.25 m.
   Diameter.—Average 5 mm.
   Internode length.—35 mm.
   Shape.—Rounded.
   Color.—146A when young turning to 152D when mature.
   Strength.—Strong.
   Pubescence.—Absent.
   Pinching.—Yes.
Foliage:
   Texture.—Leathery.
   Leaf arrangement.—Alternate.
   Compound or single.—Single.
   Quantity of leaves per lateral branch.—Varies between 2 and 9.
   Leaf shape.—Orbicular.
   Leaf apex.—Rounded.
   Leaf base.—Obtuse.
   Leaf length.—Average 70 mm in length.
   Leaf width.—Average 55 mm in width.
   Pubescence.—Absent.
   Leaf margin.—Entire.
   Young leaf color (upper surface).—144A.
   Young leaf color (lower surface).—144B.
   Mature leaf color (upper surface).—144A.
   Mature leaf color (lower surface).—144B.
   Vein color (both surfaces).—N144A.
   Petiole length.—11 mm.
   Petiole diameter.—3 mm.
   Petiole color.—N144A.
   Durability of foliage to stress.—Moderate to high.
Flowers: Flowers have not been observed.
Disease and insect resistance: Plants of the new Griselinia have not been observed for disease or insect resistance.

1. A new and distinct variety of Griselinia plant named ‘Green Hedgemaster’ as described and illustrated.
